(19) United States
(12) Patent Application Publication
(10) Pub. No.: US 2015/0257832 A1
(43) Pub. Date: Sep. 17, 2015

(54) LASER ABLATION DEVICE

(71) Applicant: OLYMPUS CORPORATION, Tokyo (JP)

(72) Inventor: Sadao EBATA, Tokyo (JP)

(73) Assignee: OLYMPUS CORPORATION, Tokyo (JP)

(21) Appl. No.: 14/687,100

(22) Filed: Apr. 15, 2015

Related U.S. Application Data
(63) Continuation of application No. PCT/JP2013/074632, filed on Sep. 12, 2013.

(30) Foreign Application Priority Data
Nov. 13, 2012 (JP) 2012-249519

(51) Int. Cl. A61B 18/24

(52) U.S. Cl. CPC A61B 18/24; A61B 2018/00595

(57) ABSTRACT

Local excessive laser radiation is prevented, and uniform laser radiation is performed in a target treatment region. Provided is a laser ablation device including: a light source that emits laser light for cauterizing an affected area; a fiber that is provided in an insertion portion and that guides the laser light emitted from the light source to radiate the laser light from an insertion-portion distal end; and a first drive unit that is provided on the fiber and that vibrates the fiber with a first period.

### CROSS-REFERENCE TO RELATED APPLICATIONS

This is a continuation of International Application PCT/JP2013/074632, with an international filing date of Sep. 12, 2013, which is hereby incorporated by reference herein in its entirety. This application claims the benefit of Japanese Patent Application No. 2012-249519, filed on Nov. 13, 2012, the content of which is incorporated herein by reference.

### TECHNICAL FIELD

The present invention relates to a laser ablation device.

### BACKGROUND ART

In the conventional art, a laser ablation catheter with which laser light is radiated onto affected tissue from an insertion portion that emits laser light having high-density energy, thus cauterizing the affected tissue, has been known (for example, Japanese Unexamined Patent Application, Publication No. Hei 7-8502). The laser ablation catheter is used mainly to perform arrhythmia treatment and has the advantage, for patients, that only a minimum region into which the laser ablation catheter can be inserted needs to be incised, thereby making it possible to cauterize an affected area, which allows minimally invasive surgery to be performed.

### CITATION LIST

### Patent Literature

### SUMMARY OF INVENTION

### Technical Problem

With the above-described laser ablation catheter, because laser light having high energy is radiated in a linear fashion, the laser cauterization performance at the affected area is high. An operator manually vibrates an insertion portion and operates the laser ablation catheter so as to prevent laser light from being locally and excessively radiated, this requires a highly-skilled operation. Particularly in a narrow space in the pericardium, manipulations performed by the operator at an insertion-portion base end to be transferred to an insertion-portion distal endare limited. Although the amount of light, the radiation region, and the radiation time are specified for laser radiation for treatment, because the density of laser radiation differs depending on the manipulation route, uneven radiation occurs.

The present invention is a laser ablation device that prevents local excessive laser radiation and that performs uniform laser radiation in a target treatment region.

### Solution to Problem

According to one aspect, the present invention provides a laser ablation device including: a light source that emits laser light for cauterizing an affected area; a fiber that is provided in an insertion portion and that guides the laser light emitted from the light source to radiate the laser light from an insertion-portion distal end; and a first drive unit that is provided on the fiber and that vibrates the fiber with a first period.

In the above-described aspect, it is preferable to further include a second drive unit that vibrates the fiber with a second period.

It is possible to set the first period and the second period to different periods or also to the same period.

In the above-described aspect, it is preferable that the amplitude produced by the second drive unit be larger than the amplitude produced by the first drive unit.

In the above-described aspect, it is preferable that the first drive unit be provided closer to a distal end of the fiber than the second drive unit; and the second period be longer than the first period.

In the above-described invention, it is preferable that the first drive unit be provided closer to a distal end of the fiber than the second drive unit; and the second period be a period n times (n is an integer) the first period.

In the above-described aspect, it is preferable that the fiber be made to perform rotational motions by the first drive unit and the second drive unit; and the number of rotations of the fiber due to the first drive unit be faster than the number of rotations of the fiber due to the second drive unit.

The first drive unit and the second drive unit allow the fiber to perform a resonant motion, raster scanning, spiral scanning, and scanning obtained by combining different types of scanning.

It is preferable that further including one or more other drive units for periodically vibrating the fiber.

### BRIEF DESCRIPTION OF DRAWINGS

FIG. 1 is a view showing the overall configuration of a laser ablation device according to a first embodiment of the present invention.

FIGS. 2A to 2C show an insertion portion of the laser ablation device according to the first embodiment of the present invention: FIG. 2A is a view showing the overall insertion portion; FIG. 2B is a view showing a state in which a shaft and a fiber are fixed; and FIG. 2C is a cross-sectional view cut along the line A-A′ in FIG. 2B.

FIG. 3 is a view showing the overall configuration of a laser ablation device according to a second embodiment of the present invention.

FIGS. 4A to 4D show an insertion portion of the laser ablation device according to the second embodiment of the present invention: FIG. 4A is a view showing the overall insertion portion; FIG. 4B is a view showing a state in which a shaft and a fiber are fixed; FIG. 4C is a cross-sectional view cut along the line A-A′ in FIG. 4B; and FIG. 4D is a cross-sectional view cut along the line B-B′ in FIG. 4B.

FIGS. 5A and 5B show example laser-light radiation trajectories produced by the fiber of the laser ablation device according to the second embodiment of the present invention.

FIG. 6 is a view showing the overall configuration of a laser ablation device according to a third embodiment of the present invention.

FIG. 7 is a view showing an insertion portion of the laser ablation device according to the third embodiment of the present invention.

FIGS. 8A to 8C show example laser-light radiation trajectories produced by a fiber of the laser ablation device according to the third embodiment of the present invention.

FIG. 9 is a view showing an insertion portion of a laser ablation device according to a modification of the third embodiment of the present invention.

FIGS. 10A to 100 show example laser-light radiation trajectories produced by a fiber of the laser ablation device according to the modification of the third embodiment of the present invention.

FIGS. 11A and 11B show other examples of insertion portions of laser ablation devices according to the present invention.

FIGS. 12A to 12C show example laser-light radiation trajectories produced by fibers of the laser ablation devices shown in FIGS. 11A and 11B.

### DESCRIPTION OF EMBODIMENTS

### First Embodiment

A laser ablation device 10 according to a first embodiment of the present invention will be described below with reference to the drawings. The laser ablation device 10 of this embodiment radiates laser light from an insertion portion, to be described later, onto affected tissue to cauterize the affected tissue, thereby performing treatment for arrhythmia etc., and includes an insertion portion 11 and a main portion 12.

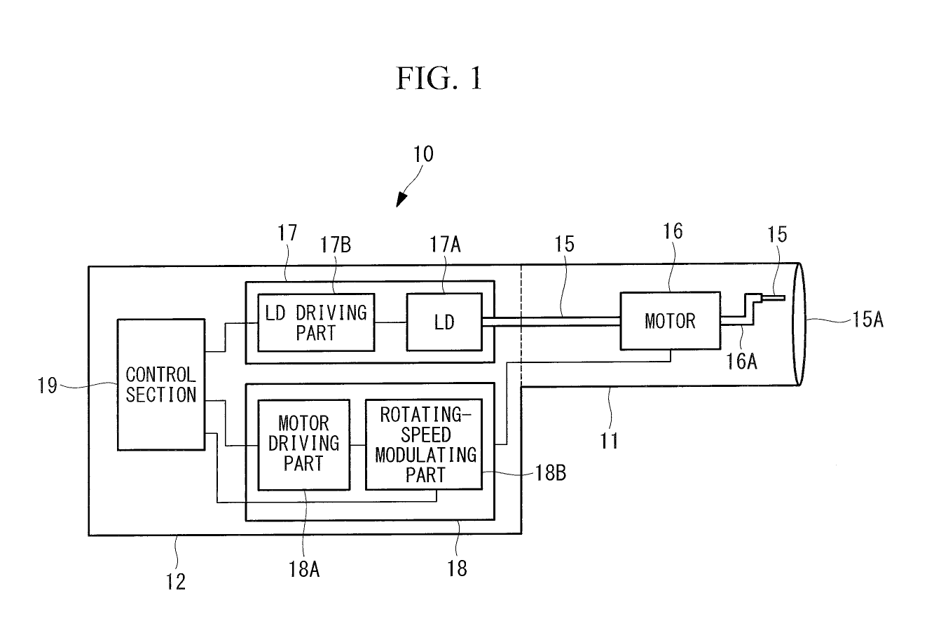

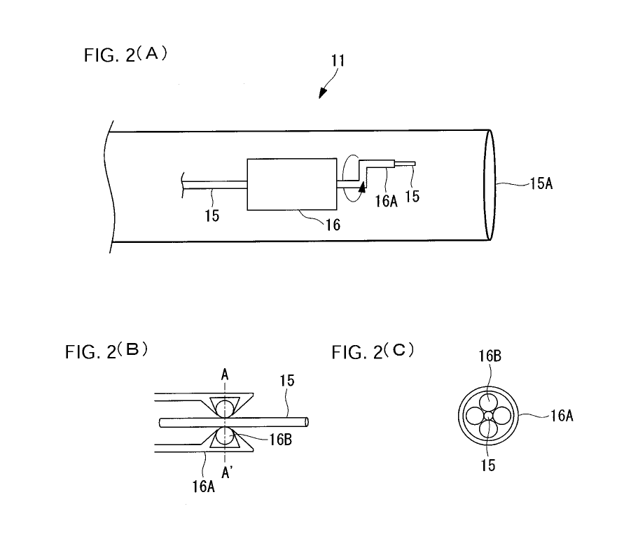

As shown in FIGS. 1 and 2, the insertion portion 11 to be inserted into the body of patients is a long bendable pipe conduit and includes a fiber 15 that guides laser light emitted from a light source, to be described later, and that radiates the laser light from an insertion-portion distal end and a motor 16 that is provided on the fiber 15 to vibrate the fiber 15 with a predetermined period. Specifically, the fiber 15 is provided integrally with a shaft 16A of the motor 16 and guides laser light while rotating in conjunction with rotation of the motor 16.

Specifically, the shaft 16A has a hollow structure, and the fiber 15 passes through the shaft 16A. The shaft 16A of the motor 16 has a bent portion, so that the output of the motor 16 is made to be eccentric with respect to the axis of rotation. As shown in FIG. 2C, four ball bearings 16B are disposed in a distal end of the shaft 16A at equal-spaced intervals, the fiber 15 is in contact with the shaft 16A via the ball bearings 16B, and thus the fiber 15 is fixed to the shaft 16A. A lens 15A through which laser light emitted from an emitting end of the fiber 15 is transmitted is provided on a distal end surface of the insertion portion 11.

The main portion 12 includes a light source section 17, a vibration control section 18 that controls the vibration of the fiber 15, and a control section 19 that controls the light source section 17 and the vibration control section 18.

The light source section 17 includes an LD (laser diode) 17A that serves as the light source, which emits laser light for cauterizing an affected area, and an LD driving part 17B that drives the LD 17A.

The vibration control section 18 has a motor driving part 18A that rotationally drives the motor 16 and a rotating-speed modulating part 18B that appropriately modulates the rotating speed of the motor 16.

The operation of the thus-configured laser ablation device 10 will be described below.

The distal end of the insertion portion 11 of the laser ablation device 10 is inserted up to the vicinity of an affected area. In this state, when power is supplied from the LD driving part 17B to the LD 17A based on a control signal sent from the control section 19, laser light is emitted from the LD 17A and enters an incident end of the fiber 15 that is located at a base end of the insertion portion 11. The laser light is guided by the fiber 15 to the distal end of the fiber 15 and is radiated from the emitting end of the fiber 15 onto the affected area via the lens 15A, which is provided at the distal end of the insertion portion 11.

At this time, the fiber 15 is provided integrally with the shaft 16A of the motor 16 so as to guide the laser light while rotating in conjunction with rotation of the motor 16. Furthermore, because the shaft 16A of the motor 16 makes the output of the motor 16 eccentric with respect to the axis of rotation, when the motor 16 is rotationally driven by the motor driving part 18A, the laser light emitted from the fiber 15 is radiated onto the affected area while tracing a circular trajectory corresponding to the eccentric position of the shaft 16A.

As described above, according to this embodiment, rotation of the motor 16 vibrates the fiber 15, which emits laser light, thereby making it also possible to vibrate the laser-light radiation trajectory, thus preventing laser light from being locally radiated onto the affected area and allowing uniform laser-light radiation while expanding the radiation region.

### Second Embodiment

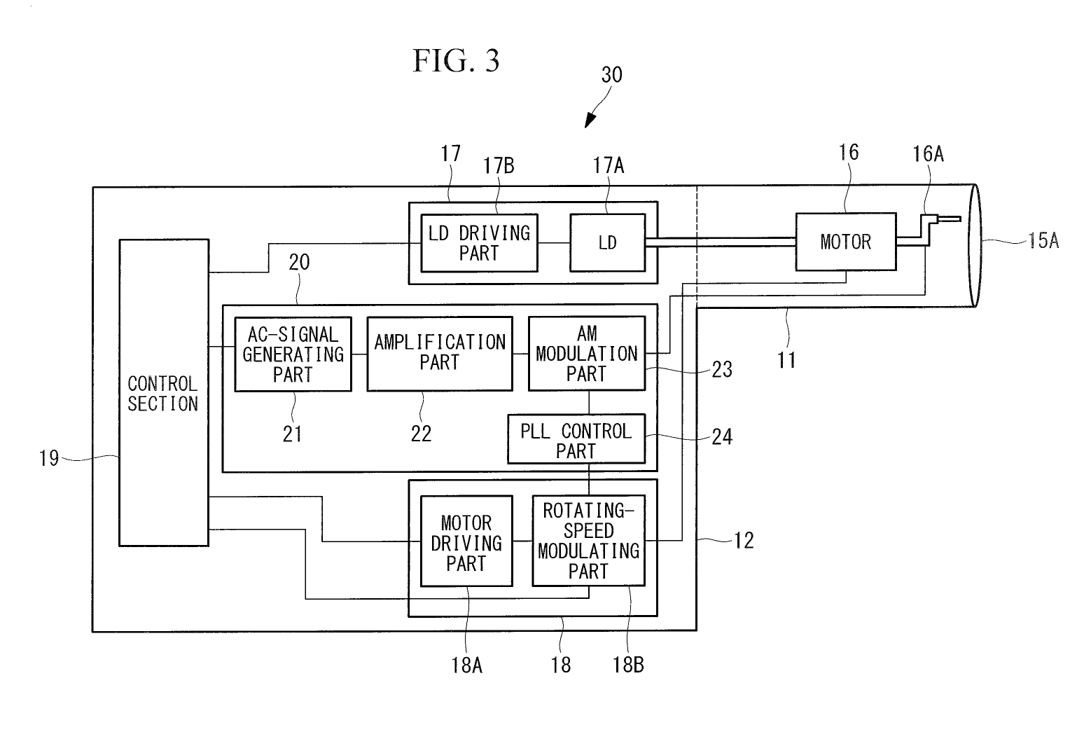

Next, a laser ablation device 30 according to a second embodiment of the present invention will be described below with reference to the drawings. In this embodiment, identical reference signs are assigned to the same components as those in the above-described first embodiment, and a description thereof will be omitted. This embodiment mainly differs from the first embodiment in that piezoelectric elements 15B are provided symmetrically in four directions around the axis of the output end of the shaft 16A, as shown in FIG. 3.

Therefore, the main portion 12 further includes a piezoelectric-element control section 20 that controls the piezoelectric elements, and the control section 19 controls the light source section 17, the vibration control section 18, and the piezoelectric-element control section 20.

The piezoelectric-element control section 20 includes an AM modulation part 23 that supplies electric power to the piezoelectric elements 15B, a PLL control part 24 that adjusts the phases of modulated signals output from the AM modulation part 23 and the number of rotations of the motor 16, an AC-signal generating part 21 that generates AC signals to be supplied to the AM modulation part 23, and an amplification part 22 that amplifies the AC signals output from the AC-signal generating part 21.

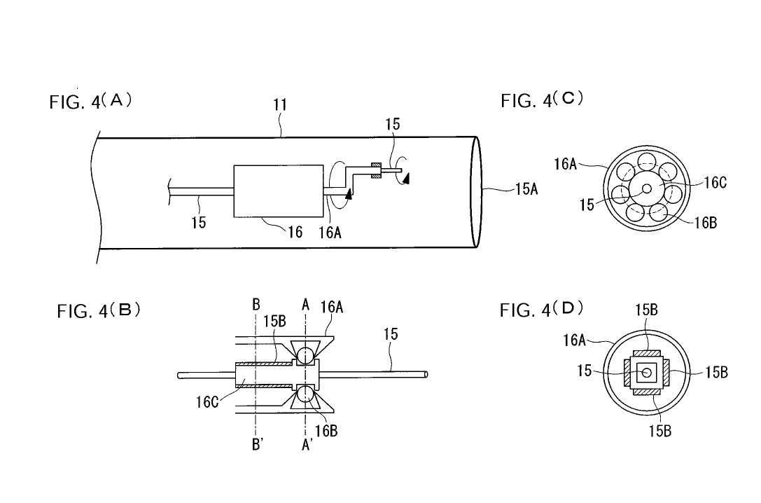

As shown in FIGS. 4A to 4D, the fiber 15 is provided in the hollow shaft 16A, and the distal end of the fiber 15 is fixed to the shaft 16A by ball bearings 16B that are provided via an elastic member 16C. Contact points of the ball bearings 16B are located at the position of a node of a vibration of the elastic member. Because the piezoelectric elements 15B are provided symmetrically in four directions around the axis of the fiber 15 via the elastic member 16C and are composed of X-axis-driving piezoelectric elements and Y-axis-driving piezoelectric elements, the phases of the AC signals supplied from the AC-signal generating part 21 to the X-axis-driving piezoelectric elements and the Y-axis-driving piezoelectric elements are shifted by 90 degrees.

Furthermore, the modulated signals output from the AM modulation part 23 and the rotating speed of the motor 16 are individually controlled by the PLL control part 24 so as to establish a relationship between frequency division and multiplication.

The operation of the thus-configured laser ablation device will now be described.

AC signals generated by the AC-signal generating part 21 are amplified by the amplification part 22 and are AM-modulated at the AM modulation part 23. The frequencies of the voltage and the current to be applied to the piezoelectric elements 15B are made to match the resonance frequency of a vibration of the fiber 15. When the modulated signals output from the AM modulation part 23 are supplied to the piezoelectric elements 15B, the piezoelectric elements 15B vibrate due to the piezoelectric effect, thus vibrating the shaft 16A. The vibration is transferred to make the fiber 15 resonate.

In this state, when the LD driving part 17B supplies predetermined power to the LD 17A based on a control signal of the control section 19, the LD 17A emits laser light toward the emitting end of the fiber 15. The emitted laser light is radiated onto an affected area from the insertion-portion distal end via the fiber 15.

At this time, as described above, because the motor 16 is driven, thereby rotating the shaft 16A, and the piezoelectric elements 15B vibrate due to the piezoelectric effect, thereby vibrating the distal end of the shaft 16A, light radiated from the distal end of the insertion portion 11 traces a radiation trajectory obtained by superposing a vibration produced by the motor and a vibration produced by the piezoelectric elements 15B.

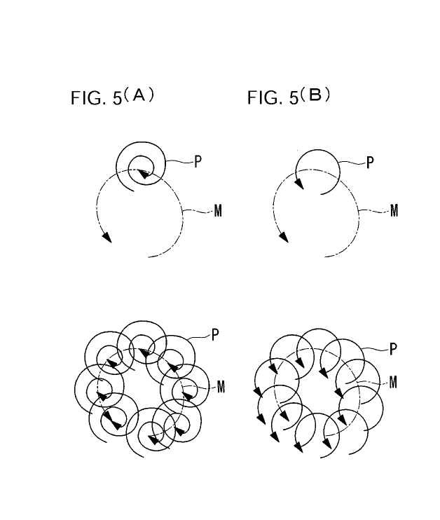

FIGS. 5A and 5B show example laser-light radiation trajectories produced by the fiber 15. FIG. 5A shows an example radiation trajectory in the case where, by setting the amplitude of a vibration produced by the piezoelectric elements 15B smaller than the amplitude of a vibration produced by the motor 16, the motor 16 roughly moves the laser light at the same time as the piezoelectric elements 15B finely move the laser light. FIG. 5A shows an example laser-light radiation trajectory in the case where the piezoelectric elements 15B vibrate the fiber 15 in a spiral pattern, and FIG. 5B shows an example laser-light radiation trajectory in the case where the piezoelectric elements 15B vibrate the fiber 15 in a circular trajectory.

In this way, according to this embodiment, by superposing the vibration produced by rotational motion of the motor 16 and the vibration produced by the piezoelectric elements 15B, it is possible to prevent the laser light from being locally radiated and also to allow more uniform laser-light radiation, which prevents damage to tissue other than the affected area. Because it is possible to avoid fixed-point radiation and to allow area radiation, the therapeutic dose can be visually perceived with observation optics, such as an endoscope.

Note that the number of rotations, the rotating speed, and the direction of rotation of the motor may be desirably set, and the amplitude of the motor may be different from or may be the same as the amplitude of the piezoelectric elements. Furthermore, in this embodiment, although a description has been given of a case in which the frequencies of the voltage and the current to be applied to the piezoelectric elements 15B are made to match the resonance frequency of the vibration of the fiber 15, the frequencies are not necessarily resonant and may be non-resonant.

### Third Embodiment

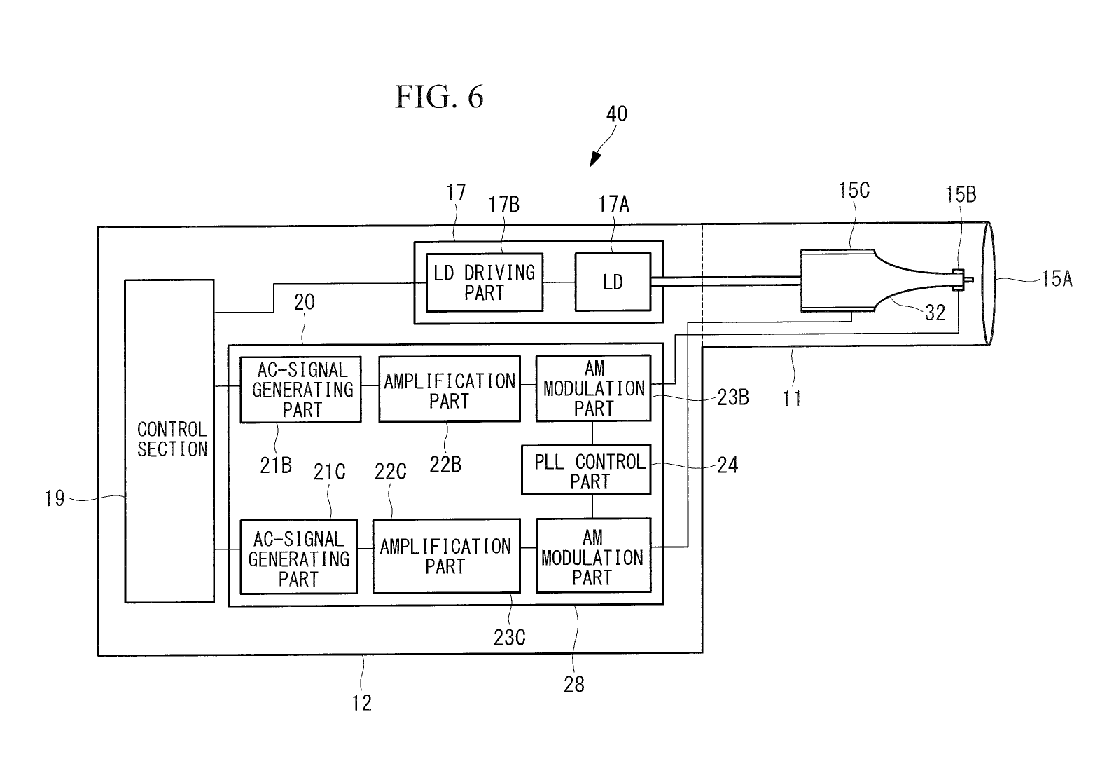

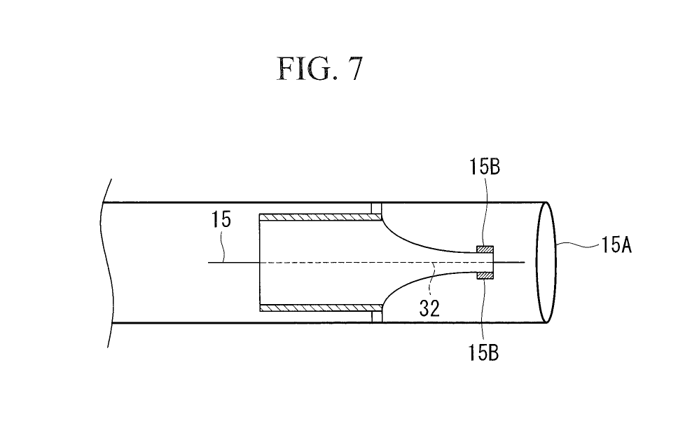

Next, a laser ablation device 40 according to a third embodiment of the present invention will be described with reference to the drawings. In this embodiment, identical reference signs are assigned to the same components as those in the above-described second embodiment, and a description thereof will be omitted. This embodiment mainly differs from the second embodiment in that piezoelectric elements 15C are provided instead of the motor 16, as shown in FIGS. 6 and 7.

Specifically, the fiber 15 is provided with an elastic member 32 for supporting the piezoelectric elements 15B and the piezoelectric elements 15C. The piezoelectric elements 15B are provided symmetrically in four directions at a distal end of the elastic member 32, and the piezoelectric elements 15C are provided symmetrically in four directions at a base end thereof.

Therefore, the main portion 12 includes, instead of the piezoelectric-element control section 20, a piezoelectric-element control section 28 that controls the piezoelectric elements 15B and the piezoelectric elements 15C.

The piezoelectric-element control section 28 includes AM modulation parts 23B and 23C that supply electric power to the piezoelectric elements 15B and 15C, respectively, a PLL control part 24 that individually adjusts the phases of modulated signals output from the AM modulation parts 23B and 23C, AC-signal generating parts 21B and 21C that generate AC signals to be supplied to the AM modulation parts 23B and 23C, and amplification parts 22B and 22C that amplify the AC signals output from the AC-signal generating parts 21B and 21C.

AC signals generated by the AC-signal generating part 21B are amplified at the amplification part 22B and are AM-modulated at the AM modulation part 23B. Similarly, AC signals generated by the AC-signal generating part 21C are amplified at the amplification part 22C and are AM-modulated at the AM modulation part 23C. Although the modulated signals output from the AM modulation part 23B and the AM modulation part 23C have different frequencies, they are controlled at the PLL control part 24 so as to establish a relationship between frequency division and multiplication. Furthermore, the frequencies of the voltage and the current to be applied to the piezoelectric elements 15B are made to match the resonance frequency at the distal end portion of the elastic member 32, and the frequencies of the voltage and the current to be applied to the piezoelectric elements 15C are made to match the resonance frequency of the fiber 15.

The operation of the thus-configured laser ablation device will now be described. Modulated signals output from the AM modulation part 23B and the AM modulation part 23C are supplied to the piezoelectric elements 15B and 15C, respectively, and the piezoelectric elements 15B and 15C vibrate due to the piezoelectric effect based on the modulated signals. The vibrations are transferred via the elastic member 32 to vibrate the fiber 15.

In this state, when the LD driving part 17B supplies predetermined power to the LD 17A based on a control signal output from the control section 19, the LD 17A emits laser light toward the incident end of the fiber 15. The emitted laser light is emitted from the distal end of the insertion portion 11 via the fiber 15.

At this time, because the piezoelectric elements 15B and 15C vibrate the fiber 15, the laser light emitted from the distal end of the insertion portion 11 traces a radiation trajectory obtained by superposing a vibration produced by the piezoelectric elements 15B and a vibration produced by the piezoelectric elements 150.

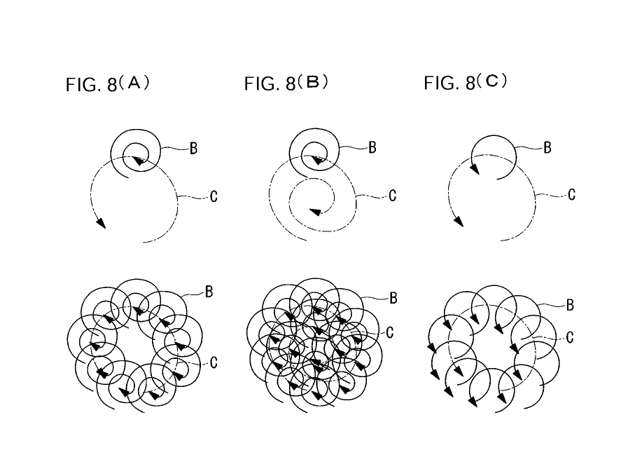

FIGS. 8A to 80 show example laser-light radiation trajectories produced by the fiber 15. FIGS. 8A to 8C show example radiation trajectories in the case where, by setting the amplitude of a vibration produced by the piezoelectric elements 15B smaller than the amplitude of a vibration produced by the piezoelectric elements 15C, the piezoelectric elements 15C roughly move the laser light at the same time as the piezoelectric elements 15B finely move the laser light. FIG. 8A shows an example laser-light radiation trajectory in the case where the piezoelectric elements 15B vibrate the fiber 15 in a spiral pattern at the same time as the piezoelectric elements 15C vibrate the fiber 15 in a circular trajectory, and FIG. 8B shows an example laser-light radiation trajectory in the case where the piezoelectric elements 15B vibrate the fiber 15 in the same way as in FIG. 8A, and the piezoelectric elements 15C vibrate the fiber 15 in a spiral pattern. FIG. 8C shows an example laser-light radiation trajectory in the case where both the piezoelectric elements 15B and 15C vibrate the fiber 15 in a circular trajectory.

In this way, according to this embodiment, the vibration produced by the piezoelectric elements 15B and the vibration produced by the piezoelectric elements 15C are transferred to the fiber 15 via the elastic member 32, and the vibration produced by the piezoelectric elements 15B and the vibration produced by the piezoelectric elements 15C are superposed, thereby making it possible to prevent the laser light from being locally radiated and also to allow more uniform laser-light radiation, which prevents damage to tissue other than the affected area. Because it is possible to avoid fixed-point radiation and to allow area radiation, the therapeutic dose can be visually perceived with observation optics, such as an endoscope. Because the variable range of the radiation region is wide, it is possible to respond flexibly to different treatment regions.

### Modification of Third Embodiment

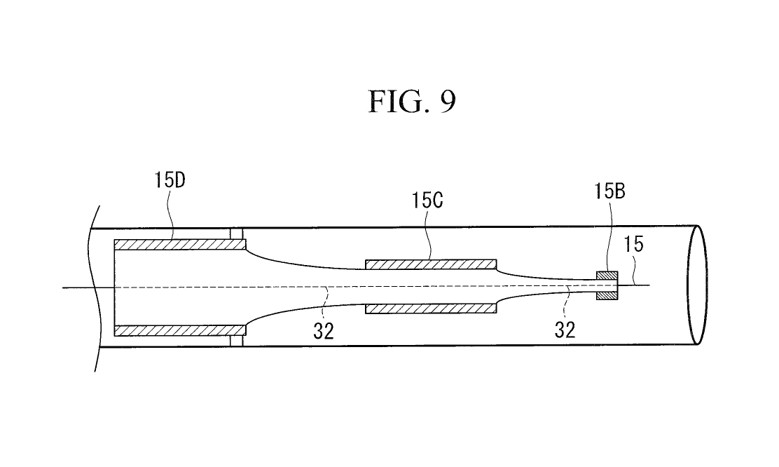

Next, a laser ablation device according to a modification of the third embodiment of the present invention will be described with reference to the drawings. In this modification, identical reference signs are assigned to the same components as those in the above-described third embodiment, and a description thereof will be omitted. This embodiment mainly differs from the third embodiment in that a so-called three-stage structure in which piezoelectric elements are provided at three places in the axial direction of the elastic member 32 is built, as shown in FIG. 9.

Specifically, the fiber 15 is provided with an elastic member 32 for supporting the piezoelectric elements 15B, the piezoelectric elements 15C, and piezoelectric elements 15D. The elastic member 32 has the piezoelectric elements 15B provided symmetrically in four directions at the distal end, the piezoelectric elements 15C provided symmetrically in four directions closer to the base end than the piezoelectric elements 15B, and the piezoelectric elements 15D provided symmetrically in four directions at the base end.

Therefore, as in the above-described third embodiment, the main portion includes, instead of the piezoelectric-element control section 20, a piezoelectric-element control section 28 that controls the piezoelectric elements 15B, the piezoelectric elements 15C, and the piezoelectric elements 15D, and the piezoelectric-element control section 28 includes AM modulation parts that supply electric power to the piezoelectric elements 15B, 15C, and 15D, a PLL control part that individually adjusts the phases of modulated signals output from the AM modulation parts, AC-signal generating parts that generate AC signals to be supplied to the AM modulation parts, and amplification parts that amplify the AC signals output from the AC-signal generating parts.

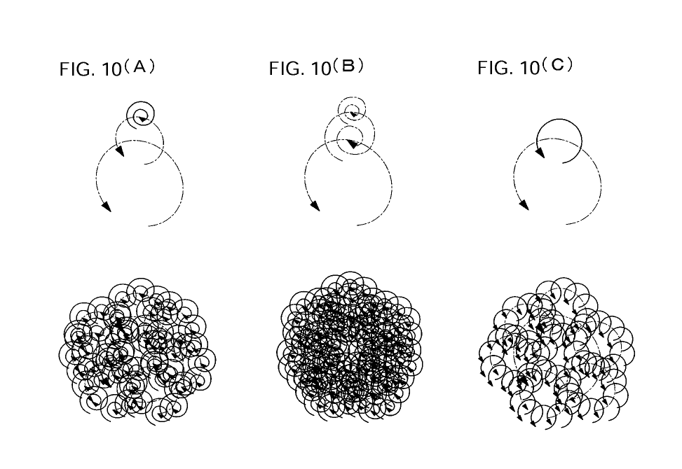

FIGS. 10A to 10C show example laser-light radiation trajectories produced by the fiber 15 in the case where the piezoelectric elements 15B, 15C, and 15D are provided at three places in the axial direction of the fiber, as described above. FIGS. 10A to 10C show example radiation trajectories in the case where, by setting the amplitude of a vibration produced by the piezoelectric elements that are provided closer to the distal end of the fiber 15 to be smaller, the piezoelectric elements that are provided closer to the base end roughly move the laser light at the same time as the piezoelectric elements that are provided closer to the distal end finely move the laser light. In particular, FIG. 10A shows an example laser-light radiation trajectory in the case where the piezoelectric elements 15B vibrate the fiber 15 in a spiral pattern at the same time as the piezoelectric elements 15C and 15D vibrate the fiber 15 in a circular trajectory. FIG. 10B shows an example laser-light radiation trajectory in the case where the piezoelectric elements 15D vibrate the fiber 15 in a circular trajectory at the same time as the piezoelectric elements 15B and 15C vibrate the fiber 15 in a spiral pattern. FIG. 10C shows an example laser-light radiation trajectory in the case where all of the piezoelectric elements 15B, 15C, and 15D vibrate the fiber 15 in a circular trajectory.

In this way, according to this embodiment, the vibrations produced by the piezoelectric elements 15B, 15C, and 15D are transferred to the fiber 15 via the elastic member 32, and the vibrations produced by the piezoelectric elements 15B, 15C, and 15D are superposed, thereby making it possible to prevent the laser light from being locally radiated and also to allow more uniform laser-light radiation, which prevents damage to tissue other than the affected area. Because it is possible to avoid fixed-point radiation and to allow area radiation, the therapeutic dose can be visually perceived with observation optics, such as an endoscope. Because the variable range of the radiation region is wide, it is possible to respond flexibly to different treatment regions.

Note that, in the above-described embodiments, although piezoelectric elements are used as a means for producing a vibration, such means is not necessarily limited to the piezoelectric elements and can be electromagnetic vibration elements, for example.

Furthermore, although the third embodiment is provided with a two-stage structure that has a drive unit in which the piezoelectric elements 15B produce a vibration and a drive unit in which the piezoelectric elements 15C produce a vibration, and the modification of the third embodiment is provided with a three-stage structure that has three drive units in each of which the piezoelectric elements produce a vibration, a structure having four or more stages may be provided, and every possible means that can vibrate the fiber, such as motors, piezoelectric elements, and electromagnetic vibration elements, can be used alone or in appropriate combinations, as drive units.

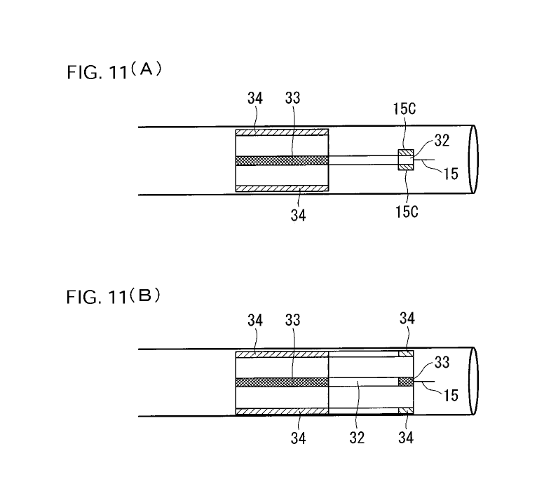

For example, as shown in FIGS. 11A and 11B, an electromagnetic vibration element 35 has a permanent magnet 33 that is disposed on the axis of the elastic member 32, which transfers a vibration to the fiber 15, and a coil 34 that is provided so as to surround the permanent magnet 33. When the thus-configured electromagnetic vibration element 35 is used, it is possible to build a structure in which the electromagnetic vibration element 35 is provided closer to the base end of the fiber 15, and the piezoelectric elements 15C are provided closer to the distal end thereof, as shown in FIG. 11A, or a structure in which the electromagnetic vibration element 35 is provided closer to the base end of the fiber 15 and also closer to the distal end thereof, as shown in FIG. 11B.

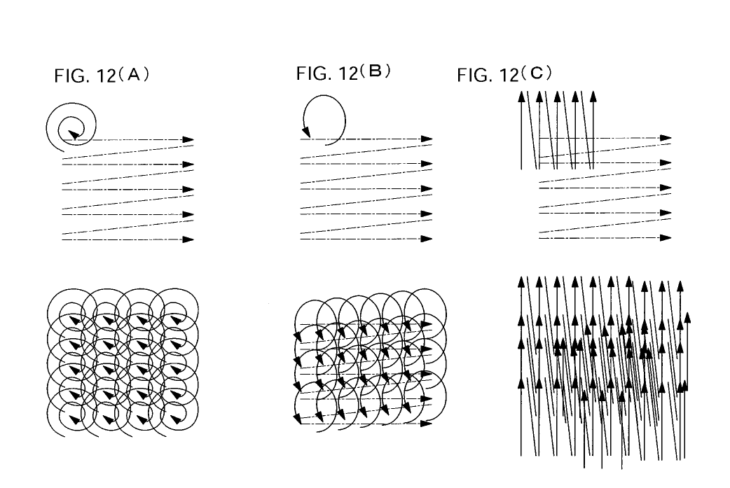

Then, when current is supplied to the coil, the permanent magnet vibrates due to electromagnetic induction, and this vibration vibrates the distal end of the fiber 15 via the elastic member. Because the electromagnetic vibration element 35 can perform raster scanning, when the piezoelectric elements are provided closer to the distal end of the fiber 15, as shown in FIG. 11A, the raster scanning can be combined with a vibration produced by rotation of the piezoelectric elements, as shown in FIGS. 12A and 12B. Furthermore, when the electromagnetic vibration element 35 is provided closer to the base end of the fiber 15 and also closer to the distal end thereof, as shown in FIG. 11B, if both of the electromagnetic vibration elements 35 perform raster scanning, a scan trajectory shown in FIG. 12C can be obtained. In either case, laser light can be prevented from being locally radiated.

### REFERENCE SIGNS LIST

10, 30, 40 laser ablation device
11 insertion portion
15 fiber
15B piezoelectric elements
15C piezoelectric elements
16 motor
16A shaft
17A light source
18 vibration control section
19 control section
20, 28 piezoelectric-element control section

1. A laser ablation device comprising:
a light source that emits laser light for cauterizing an affected area;
a fiber that is provided in an insertion portion and that guides the laser light emitted from the light source to radiate the laser light from an insertion-portion distal end; and
a first drive unit that is provided on the fiber and that vibrates the fiber with a first period.

2. A laser ablation device according to claim 1, further comprising a second drive unit that vibrates the fiber with a second period.

3. A laser ablation device according to claim 2, wherein the amplitude produced by the second drive unit is larger than the amplitude produced by the first drive unit.

4. A laser ablation device according to claim 2,
wherein the first drive unit is provided closer to a distal end of the fiber than the second drive unit; and
the second period is longer than the first period.

5. A laser ablation device according to one of claim 2,
wherein the first drive unit is provided closer to a distal end of the fiber than the second drive unit; and
the second period is a period n times (n is an integer) the first period.

6. A laser ablation device according to one of claim 2,
wherein the fiber is made to perform rotational motions by the first drive unit and the second drive unit; and
the number of rotations of the fiber due to the first drive unit is faster than the number of rotations of the fiber due to the second drive unit.

7. A laser ablation device according to one of claim 1, wherein the fiber is made to perform a resonant motion.

8. A laser ablation device according to one of claim 1, wherein the fiber is made to perform raster scanning.

9. A laser ablation device according to one of claim 1, wherein the fiber is made to perform spiral scanning.

10. A laser ablation device according to one of claim 1, further comprising one or more drive units that vibrate the fiber periodically.
